(19) United States
(12) Patent Application Publication
(10) Pub. No.: US 2021/0348109 A1
(43) Pub. Date: Nov. 11, 2021

(54) SYSTEMS AND METHODS FOR ELECTROPORATION

(71) Applicants: Frank MODICA, Silver Spring, MD (US); Nicholas CHOPAS, Germantown, MD (US); James William LUTHER, Milan (IT); Bertold ENGLER, Altensteig (DE); Andrea BESANA, Seveso (IT); Thomas Alan PEACH, Milan (IT)

(72) Inventors: Frank MODICA, Silver Spring, MD (US); Nicholas CHOPAS, Germantown, MD (US); James William LUTHER, Milan (IT); Bertold ENGLER, Altensteig (DE); Andrea BESANA, Seveso (IT); Thomas Alan PEACH, Milan (IT)

(73) Assignee: MaxCyte, Inc

(21) Appl. No.: 17/317,797

(22) Filed: May 11, 2021

Related U.S. Application Data
(60) Provisional application No. 63/023,093, filed on May 11, 2020.

(51) Int. Cl. C12M 1/42; C12M 1/32; C12M 3/00

(52) U.S. Cl. CPC C12M 35/02; C12M 23/42; C12M 23/12

(57) ABSTRACT

Electroporation systems and methods are provided that include a processing assembly including a housing, a lid rotationally connectable to the housing, an opening in a top surface of the housing, an electroporation chamber below the opening in the housing, wherein the electroporation chamber comprises (i) two or more electrodes coated with an electrically conductive, non-cytotoxic material, and (ii) a gasket forming the shape of the electroporation chamber and defining the volume of one or more wells within the electroporation chamber. The system may include a docking station, the docking station comprising, a housing, a port in the housing configured to receive the processing assembly, a lid connected to the housing, one or more contacts configured to connect the docking station to an electroporation system housing.

### CROSS-REFERENCE TO RELATED APPLICATION

This application claims priority to U.S. Provisional Application No. 63/023,093, filed May 11, 2020, titled, “SYSTEMS AND METHODS FOR ELECTROPORATION,” which is incorporated herein by reference in its entirety.

### FIELD

The disclosure generally relates to systems and methods for the introduction of chemical or biological agents into living cells or cell particles or lipid vesicles.

### BACKGROUND

There exists a need for improved systems and methods for systems and methods for electroporation, as disclosed herein.

### SUMMARY

Embodiments of the present disclosure provide a processing assembly configured for use in an electroporation system. The processing assembly may include a housing, a lid connected to the housing, an opening in a top surface of the housing, an electroporation chamber below the opening of the housing, wherein the electroporation chamber comprises (i) a gasket forming the shape of the electroporation chamber and defining the volume of one or more wells within the electroporation chamber, and (ii) two or more electrodes comprising an electrically conductive, non-cytotoxic metal, wherein the two or more electrodes are positioned on opposing sides of the electroporation chamber, and wherein the processing assembly further comprises two or more buses, each connected to a single electrode.

Embodiments of the present disclosure may provide a multi-well processing assembly configured for use in an electroporation system. The multi-well processing assembly may include a housing, a lid rotationally connectable to the housing, an opening in a top surface of the housing, an electroporation chamber below the opening of the housing, wherein the electroporation chamber comprises (i) a gasket forming the shape of the electroporation chamber and defining the volume of one or more wells within the electroporation chamber, and (ii) two or more electrodes comprising an electrically conductive, non-cytotoxic metal, wherein the two or more electrodes are positioned on opposing sides of the electroporation chamber, and wherein the multi-well processing assembly further comprises two or more buses, each connected to a single electrode.

Embodiments of the present disclosure may provide a docking station configured for use in an electroporation system. The docking station may include a housing, a port in the housing configured to receive one or more processing assemblies, a lid connected to the housing, and one or more contacts configured to connect the docking station to an electroporation system.

Embodiments of the present disclosure may provide an electroporation system that includes a processing assembly configured for use in an electroporation system. The processing assembly may include a housing, a lid connected to the housing, an opening in a top surface of the housing, an electroporation chamber below the opening of the housing, wherein the electroporation chamber comprises (i) a gasket forming the shape of the electroporation chamber and defining the volume of one or more wells within the electroporation chamber, and (ii) two or more electrodes comprising an electrically conductive, non-cytotoxic metal, wherein the two or more electrodes are positioned on opposing sides of the electroporation chamber, and wherein the processing assembly further comprises two or more buses, each connected to a single electrode. The electroporation system may also include a docking station including a housing, a port in the housing configured to receive the processing assembly, a lid connected to the housing, and one or more contacts configured to connect the docking station to an electroporation system housing.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 illustrates a left, top perspective view of a processing assembly in a closed position, consistent with embodiments of the present disclosure;

FIG. 2 illustrates a left, top perspective view of the processing assembly of FIG. 1 in an open position, consistent with embodiments of the present disclosure;

FIG. 3 illustrates a rear, top, right perspective view of the processing assembly of FIG. 1 in the open position, consistent with embodiments of the present disclosure;

FIG. 4 illustrates a rear, top, right perspective view of the processing assembly of FIG. 1 in the open position, consistent with embodiments of the present disclosure;

FIG. 5 illustrates an exploded perspective view of the processing assembly of FIG. 4, consistent with embodiments of the present disclosure;

FIG. 6 illustrates an exploded perspective view of the processing assembly of FIG. 4, consistent with embodiments of the present disclosure;

FIG. 7 illustrates a top, right perspective view of the processing assembly of FIG. 1 with a label, consistent with embodiments of the present disclosure;

FIG. 8 illustrates a top, left perspective view of the processing assembly of FIG. 1 with a label, consistent with embodiments of the present disclosure;

FIG. 9 illustrates a top, right perspective view of the processing assembly of FIG. 1 with a loading device inserted, consistent with embodiments of the present disclosure;

FIG. 10 illustrates a top, right perspective view of the processing assembly of FIG. 9, with portions of the processing assembly removed from view, consistent with embodiments of the present disclosure;

FIG. 11 illustrates a top right perspective view of a tray holding processing assemblies, consistent with embodiments of the present disclosure;

FIG. 12 illustrates a front view of trays holding processing assemblies, consistent with embodiments of the present disclosure;

FIG. 13 illustrates a top right perspective view of a tray holding processing assemblies, consistent with embodiments of the present disclosure;

FIG. 14 illustrates front views of a plurality of gaskets, consistent with embodiments of the present disclosure;

FIG. 15 illustrates a top view of an array of processing assemblies and a front view of a gasket, consistent with embodiments of the present disclosure;

FIG. 16 illustrates a front view of a bag and processing apparatus consistent with embodiments of the present disclosure;

FIG. 17 illustrates a front view of a gasket, consistent with embodiments of the present disclosure;

FIG. 18 illustrates a right, top perspective view of another processing assembly in a closed position, consistent with embodiments of the present disclosure;

FIG. 19 illustrates a right, top perspective view of the processing assembly of FIG. 18 in an open position, consistent with embodiments of the present disclosure;

FIG. 20 illustrates an exploded perspective view of the processing assembly of FIG. 18, consistent with embodiments of the present disclosure;

FIG. 21 illustrates a tray holding a plurality of processing assemblies, consistent with embodiments of the present disclosure;

FIG. 22 illustrates a processing assembly, consistent with embodiments of the present disclosure;

FIG. 23 illustrates a tray for holding a plurality of processing assemblies, and a tray and tray cover for holding a plurality of processing assemblies, consistent with embodiments of the present disclosure;

FIG. 24 illustrates a tray for holding a plurality of processing assemblies, consistent with embodiments of the present disclosure;

FIG. 25 illustrates a tray for holding a plurality of processing assemblies, consistent with embodiments of the present disclosure;

FIG. 26 illustrates a tray for holding a plurality of processing assemblies, and a tray cover, consistent with embodiments of the present disclosure;

FIG. 27 illustrates electroporation systems, consistent with embodiments of the present disclosure;

FIG. 28 illustrates a docking station in an open position with a processing assembly removed, consistent with embodiments of the present disclosure;

FIG. 29 illustrates the docking station of FIG. 28 in an open position with a processing assembly inserted, consistent with embodiments of the present disclosure;

FIG. 30 illustrates the docking station of FIG. 28 in a closed position with a processing assembly inserted, consistent with embodiments of the present disclosure;

FIG. 31 illustrates a docking station in an open position, a closed position, and connected to an electroporation system, consistent with embodiments of the present disclosure;

FIG. 32 illustrates a docking station connected to an electroporation system, consistent with embodiments of the present disclosure;

FIG. 33 illustrates an electroporation device, processing assembly, docking station, trays, and a filling apparatus, consistent with embodiments of this disclosure;

FIG. 34 illustrates exemplary packaging apparatuses, consistent with embodiments of the present disclosure;

FIG. 35 illustrates exemplary packaging bags for use with an electroporation system, consistent with embodiments of the present disclosure;

FIG. 36 illustrates exemplary packaging apparatuses, consistent with embodiments of the present disclosure;

FIG. 37 illustrates exemplary packaging apparatuses, consistent with embodiments of the present disclosure;

FIG. 38 illustrates exemplary packaging apparatuses, consistent with embodiments of the present disclosure;

FIG. 39 illustrates exemplary packaging apparatuses, consistent with embodiments of the present disclosure;

FIG. 40 illustrates exemplary packaging apparatuses, consistent with embodiments of the present disclosure;

FIG. 41 illustrates exemplary packaging apparatuses, consistent with embodiments of the present disclosure;

FIG. 42 illustrates exemplary packaging apparatuses, consistent with embodiments of the present disclosure;

FIG. 43 illustrates an exemplary vessel for delivery to an electroporation system, consistent with embodiments of the present disclosure;

FIG. 44 illustrates an exemplary vessel for delivery to an electroporation system, consistent with embodiments of the present disclosure;

FIG. 45 illustrates an exemplary vessel for delivery to an electroporation system, consistent with embodiments of the present disclosure;

FIG. 46 illustrates an exemplary vessel for delivery to an electroporation system, consistent with embodiments of the present disclosure;

FIG. 47 illustrates an exemplary vessel for delivery to an electroporation system, consistent with embodiments of the present disclosure;

FIG. 48 illustrates an exemplary vessel for delivery to an electroporation system, consistent with embodiments of the present disclosure;

FIG. 49 illustrates a connection assembly that may connect a syringe;

FIG. 50 illustrates a front view of a gasket, consistent with embodiments of the present disclosure;

FIG. 51 depicts cell viability results obtained using two gaskets, under low energy and high energy electroporation settings; and

FIG. 52 depicts the percent of viable cells expressing GFP after electroporation with nucleic acid encoding GFP.

### DETAILED DESCRIPTION

As discussed in further detail below, embodiments of the present disclosure may provide systems and methods for electroporation that may include processing assemblies, trays, gaskets, docking stations, racks, packaging, and vessels for delivery to an electroporation system.

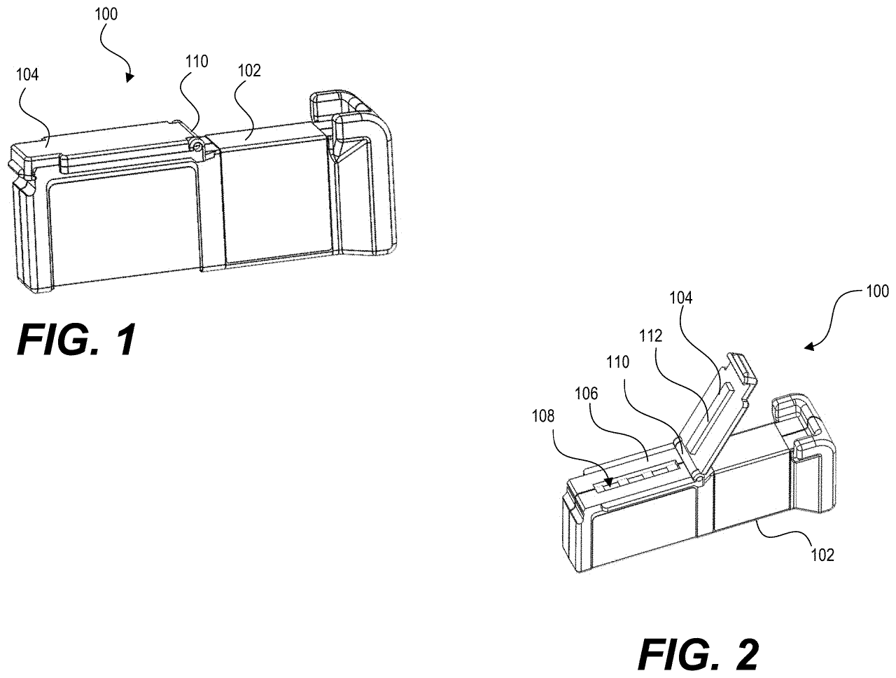

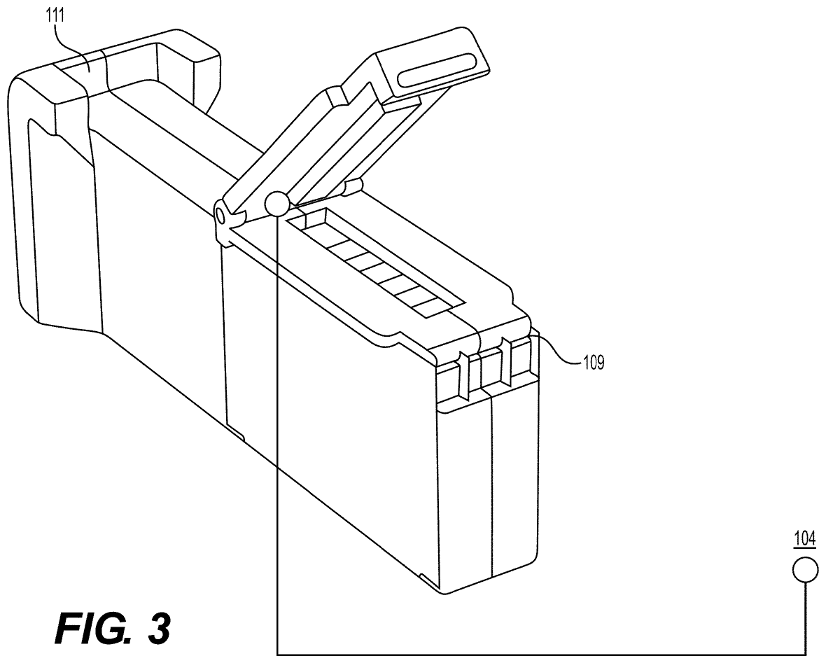

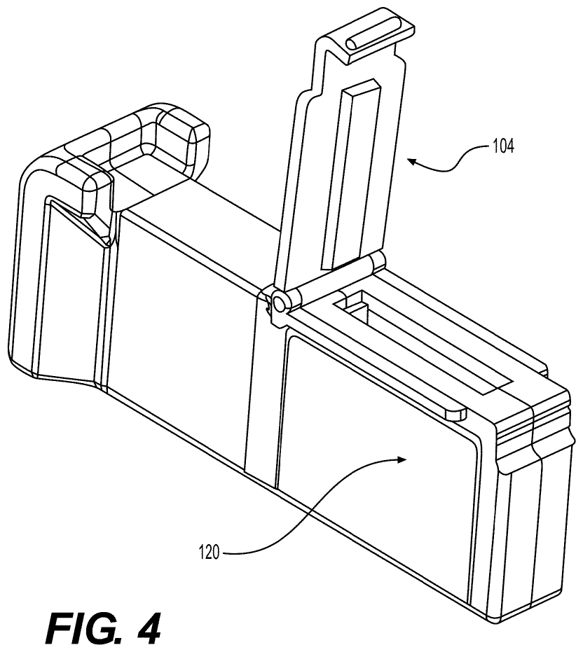

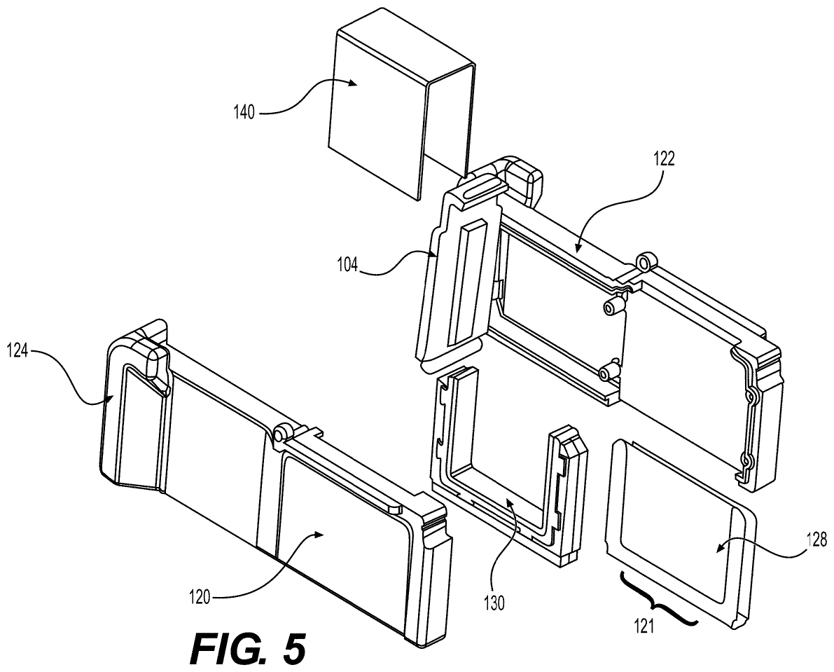

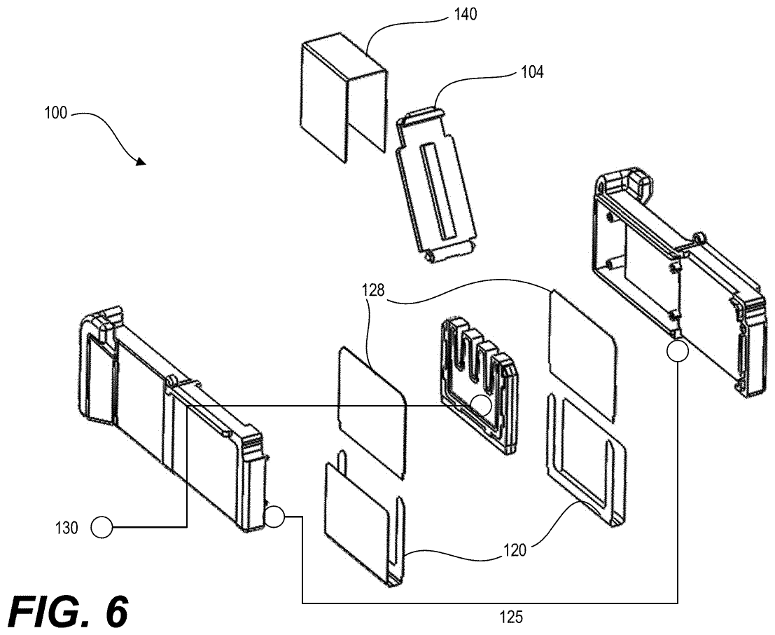

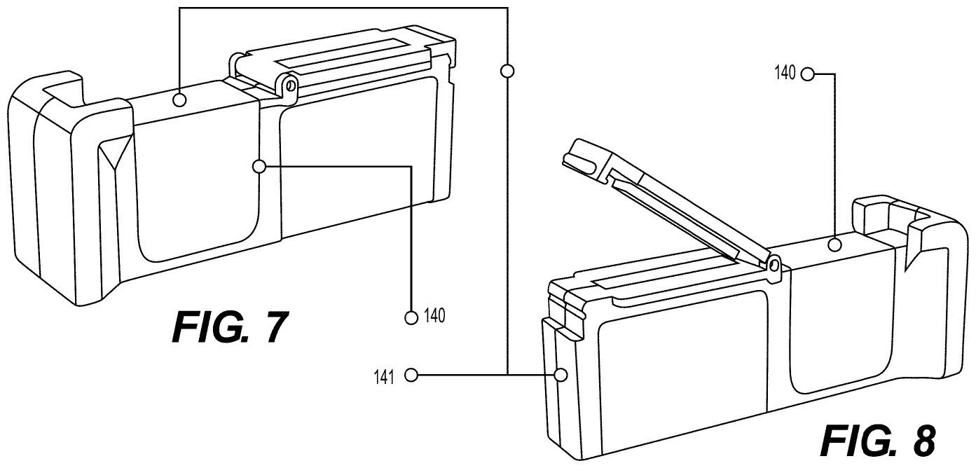

Turning now to the drawings, FIGS. 1-10 illustrate a processing assembly 100 consistent with embodiments of this disclosure. The processing assembly 100 may be provided for use in electroporation systems and devices. The processing assembly 100 may include a housing 102 and a lid 104 that covers an opening 106 to a chamber 108. In some embodiments, chamber 108 may receive samples, cultures, liquid media, etc., that may be provided to an electroporation system or device that processing assembly 100 may be compatible with.

Lid 104 may have a hinged connection 110 to the housing 102, that allows lid 104 to move between a closed position (FIG. 1) where the lid covers opening 106 and connects to housing 102, and an open position (FIG. 2) where the lid is hinged away from opening 106 and allowing opening 106 to be exposed. The hinged connection 110 of lid 104 may provide improved handling and ease-of-use of processing assembly 100. In the closed position, lid 104 may maintain sterility of processing assembly 100. In some embodiments, lid 104 may swivel about hinged connection 110 at 180° and may connect to housing 102. Some embodiments may provide lid 104 which may connect to housing 102 via an interference fit where lid 104 clips to the housing 102. For example, the interference fit may connect lid 104 to housing 102 in the closed position at connection 109 and in an open position at connection 111. The interference fit may maintain a tight seal across well(s) within chamber 108 when lid 104 is closed. Lid 104 may further include a contoured surface 112 that may connect to and cover opening 106 and maintain a sterile seal.

The chamber 108 may be an electroporation chamber that is a six-sided volume comprising a bottom and two opposing sides formed by a gasket (e.g., gasket 130) made of silicone rubber (or similar non-cytotoxic material), two parallel opposing sides formed from an electrically conductive, non-cytotoxic material (e.g., gold coated plastic film 128), and a top lid 104, made of polycarbonate (or similar non-cytotoxic plastic), which can be moved to allow dispensing materials in solution and into the chamber prior to electroporation, and aspiration of materials in solution from the chamber after electroporation.

Housing 102 may include a left handle 122 and a right handle 124 that connect to each other to form housing 102. The left handle 122 and right handle 124 may be spaced apart by pins 125 (or other features) that may be positioned opposite each other and may connect the left handle 122 and right handle 124.

Processing assembly 100 may further include two buses 120, one wrapped around the right handle 124 and one wrapped around the left handle 122. Each bus 120 comprises a thin film of electrically conductive metal. In some embodiments, the bus 120 comprises a thin film of aluminum. Processing assembly 100 may further include two or more electrodes 128. The bus 120 may be joined to the electrode 128 to form an electrode-bus assembly 121. In some embodiments, the bus 120 is joined to the electrode 128 by an adhesive layer to form an electrode-bus subassembly 121. The bus 120 may be configured to form an electrical connection between the electrode 128 inside the electroporation chamber, and the contacts in the electroporation instrument.

Processing assembly 100 may further include two or more electrodes 128 comprising an electrically conductive, non-cytotoxic metal, one to be received on the left handle 122 and the other on the right handle 124. In some embodiments, the electrically conductive, non-cytotoxic metal is aluminum, titanium, or gold. In some embodiments, the electrically conductive, non-cytotoxic metal is gold. Electrode 128 may comprise gold vacuum deposited on large rolls of plastic film that can be die cut to size and to be installed on processing assembly 100. Processing assembly 100 may include two electrodes 128 that are comprised of gold that is vacuum deposited onto a thin plastic film. The electrodes 128 may be evenly spaced apart across the chamber 108 and arranged parallel to the opposing electrode.

Processing assembly 100 may include a gasket 130 and plastic spacer that may be received in chamber 108. The gasket 130 forms in part the chamber 108 shape and determines the volume of the well(s). The gasket 130 forms liquid-tight seals of the well, and the gasket 130 may form multiple wells. The spacer may be a non-electrically conductive element that supports the shape of the gasket, maintains the distance between the electrodes 128, and maintains the parallelism of the electrodes 128. The gasket 130 may take at least one of several shapes and sizes as described in more detail below. For example, gasket 130 may be sized to receive samples of a variety of sizes including samples sized at 1000 μL, 400 μL, 100 μL, 100 μL×2, 50 μL×3, 50 μL×8, and 25 μL×3 variants, among others. In some embodiments, gasket 130 may be made of silicone rubber or other flexible materials. Processing assembly 100 may be configured for use with any one of the gasket sizes and arrangements described herein such that the processing assembly 100 may be used for any number of sized gaskets 130.

Processing assembly 100 may further include a device label 140 that extends around housing 102 away from buses 120. In some embodiments, device labels 140 may include a unique product serial number, size, instructions, logos, etc. Some embodiments may also provide for writing space 141 on an end of processing assembly 100.

Processing assembly 100 may provide several advantages including an increased volume range of samples within chamber 108 and gasket 130, an improved ease of use, and improvements in cell recovery and consistent performance. In some embodiments, gold coated plastic film 128 may provide a manufacturing cost reduction, and may allow for reaction volumes of 25-1000 microliters using a variety of gaskets.

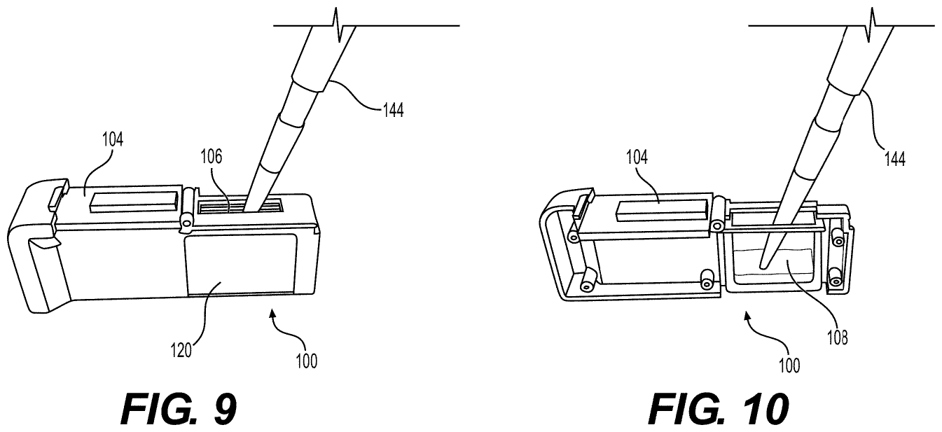

FIGS. 9 and 10 show processing assembly 100 may be configured to be filled via a loading device 144 that may be inserted into chamber 108 via opening 106 with lid 104 in the open position. Loading device 144 may fill chamber 108 with a sample for testing or for use in treating patients. Exemplary samples suitable for testing include samples comprising gene editing reagents (such as, e.g., CRISPR/Cas9 reagents, TALENs, or zinc-finger nucleases), reagents for reducing expression of one or more target proteins (such as, e.g., siRNA or other oligonucleotides suitable for reducing expression of target proteins), nucleotides encoding proteins of interest (such as, e.g., target proteins, suppressor proteins, protein antigens, one or more subunits of a multi-subunit proteins, antibodies or fragments of antibodies), or small molecule compounds. After loading device 144 provides the sample to chamber 108, loading device 144 may be removed and lid 104 may be closed to maintain sterility of sample.

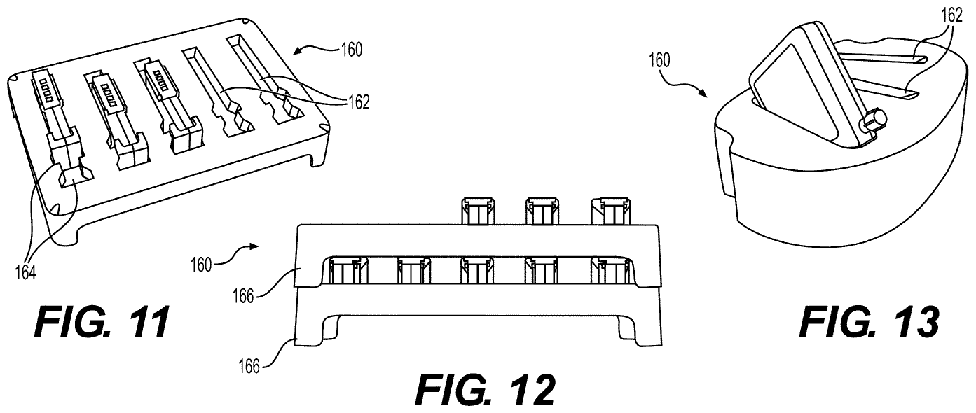

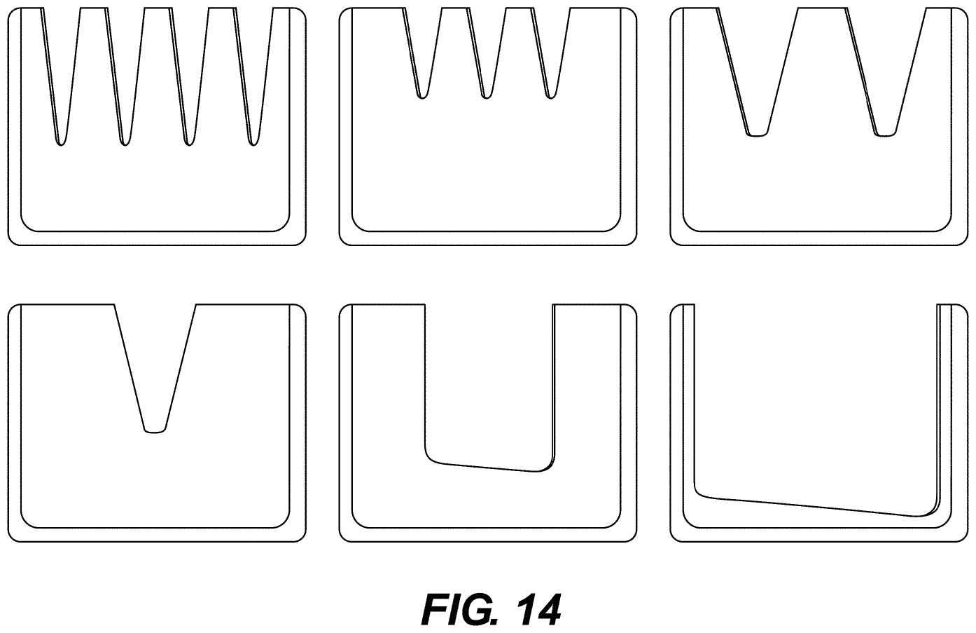

FIGS. 11-13 illustrate embodiments of the present disclosure that may also provide one or more trays 160. Trays 160 may receive one or more processing assemblies (e.g., processing assembly 100 or other processing assemblies) in slots 162 spaced apart across the tray 106. In some embodiments, trays 160 may be rectangular in shape and each slot 160 may be arranged parallel to the other slots 160. In other embodiments, tray 160 may be curved, circular, or semi-circular and may have slots 160 arranged in a radial pattern around tray 160.

Tray 160 may include one or more positions for receiving processing assemblies. In some embodiments, the tray 160 may include one or more positions 164 such that the first position and second position may allow a user to distinguish a state (e.g., complete vs. incomplete, tested vs. untested, distinguish between sample type) of the processing assembly placed in tray 160. Trays 160 may have legs 166 that may allow one or more trays 160 to be stacked on top of each other while providing clearance for the processing assemblies loaded into the tray.

Trays 160 may provide for improvements in the transportability and organization of processing assemblies and may allow for sterilization of an array of processing assemblies at once.

FIG. 14 illustrates a plurality of gaskets that could be implemented as gasket 130 within processing assembly 100 described above. Gasket 130 may be sized to receive samples of a variety of sizes including samples sized at 3×50 μL, 8×50 μL, 3×25 μL, 2×100 μL, 100 μL, 400 μL, 1 mL, among others. In some embodiments, the 400 μL and 1 mL sized gaskets may have a sloped bottom surface that may provide for improved loading and unloading of samples.

In other embodiments, the bottom surface may be flat instead of sloped.

In some embodiments, the gaskets may provide flexibility, and allow the use of a single or multi-well configuration to optimize workflow. Gaskets may also provide scalability and reduced dead volume by seamlessly shifting between small and large scale volumes on a single platform. Gaskets may also provide improved functionality where functional design maintains sterility while providing ease of use.

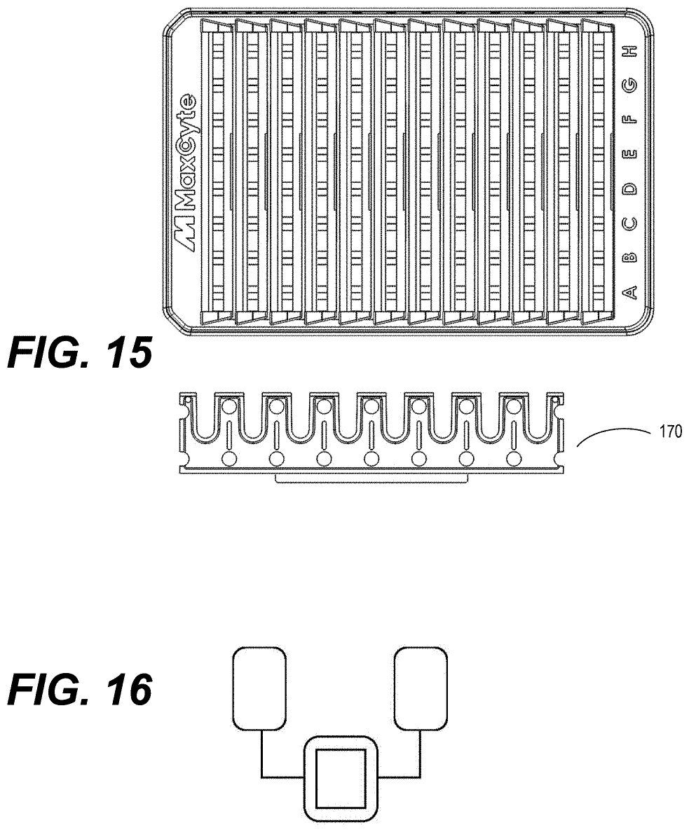

FIG. 15 illustrates a top view of an array of gaskets and a front view of a gasket, consistent with embodiments of the present disclosure, where each gasket has eight wells.

FIG. 16 illustrates a front view of a bag and processing apparatus consistent with embodiments of the present disclosure. The processing apparatus may have a V-shaped design for cell retrieval. Additionally, the processing assembly may include a 5-10 mL bag to provide a processing assembly volume between 1000 μL and 100 mL where none existed previously.

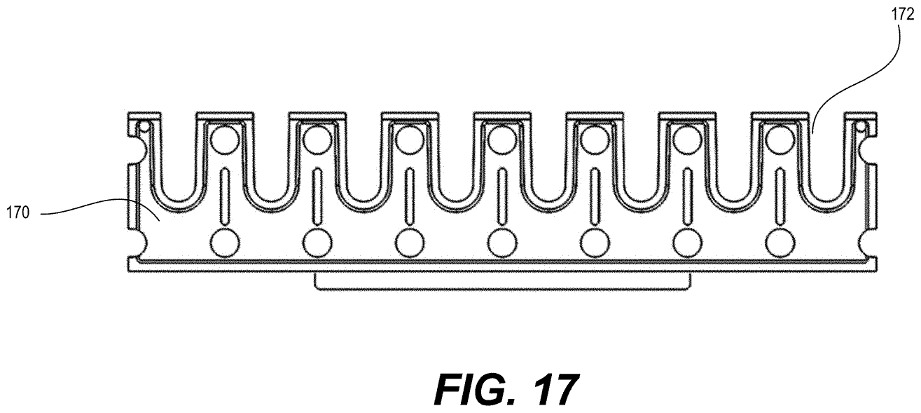

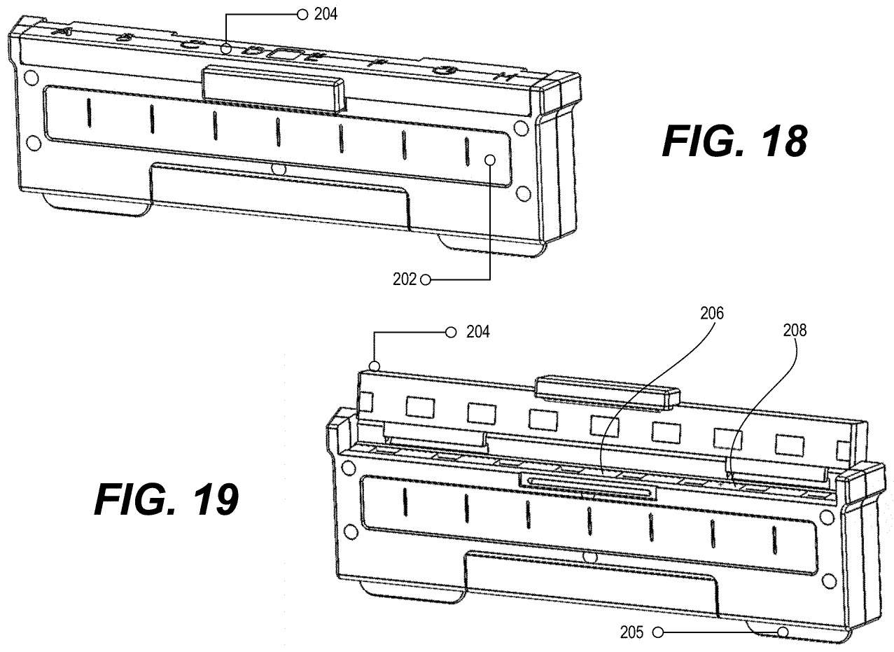

FIG. 17 illustrates a gasket 170 having eight wells 172 which may be sized for samples of 50 μL in each well 172. Gasket 170 may be configured to be received or inserted into a multi-well processing assembly 200. FIGS. 18-20 illustrate multi-well processing assembly 200 that may be configured to allow processing of multiple loaded wells (e.g., wells 172) by an electroporation system.

Multi-well processing assembly 200 may include a housing 202 with a lid 204 that extends along the length of the housing and covers an opening 206 to a chamber 208. In some embodiments, chamber 208 may receive samples, cultures, liquid media, etc., that may be provided to an electroporation system or device that processing assembly 200 may be compatible with.

Lid 204 may have a hinged connection 210 to one side of the housing 202, that allows lid 204 to move between a closed position (FIG. 18) where the lid covers opening 206 and connects to housing 202, and an open position (FIG. 19) where the lid is hinged away from opening 206 and allowing opening 206 to be exposed. In the closed position, lid 204 may maintain sterility of processing assembly 200. In some embodiments, lid 204 connected to housing 202 via an interference fit where lid 204 clips to the housing 202. In some embodiments, lid 204 may be removeable from the housing 202. In some embodiments, processing assembly 200 may have a base 205 that allows the housing 202 to stand on its own, which may provide for ease of use, loading, and stability during loading.

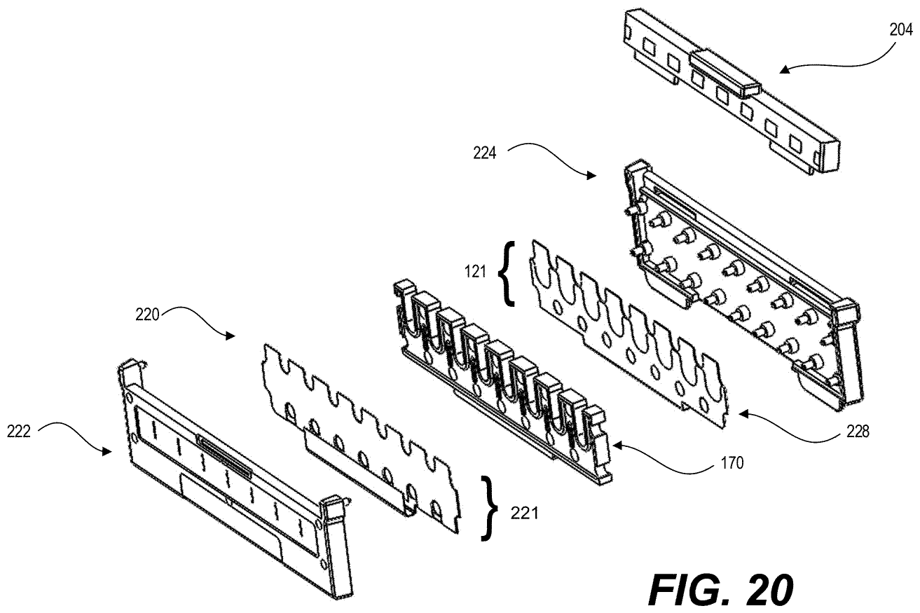

As shown in FIG. 20, housing 202 may include a left handle 222 and a right handle 224 that connect to each other to form housing 202. The left handle 222 and right handle 224 may be spaced apart by pins 225 (or other features) that may be positioned opposite each other and may connect the left handle 222 and right handle 224.

Processing assembly 200 may further include two or more electrodes 228 comprising an electrically conductive, non-cytotoxic metal, where one electrode is received on the left handle 222 and the other is received on the right handle 224. In some embodiments, the electrically conductive, non-cytotoxic metal is aluminum, titanium, or gold. In some embodiments, the electrically conductive, non-cytotoxic metal is gold. Electrode 228 may have gold vacuum deposited on large rolls of plastic film that can be die cut to size and for installation on processing assembly 200.

Processing assembly 200 may further include two buses 220, one wrapped around the right handle 224 and one wrapped around the left handle 222. Each bus 220 comprises a thin film of electrically conductive metal. In some embodiments, the bus 220 comprises a thin film of aluminum. The bus 220 forms an electrical connection between the electrode 228 inside the electroporation chamber, and the contacts in the electroporation instrument.

In some embodiments, the electrode 228 is joined to the bus 220 to form an electrode-bus subassembly 221. In some embodiments, the electrode 228 is joined to the bus 220 by an adhesive layer to form an electrode-bus subassembly 221. The processing assembly shown in FIG. 20, comprises two electrodes 228 and two buses 220 joined together to form two electrode-bus assemblies 221, wherein each bus is joined to a single electrode. The component labeled 220 in FIG. 20 corresponds to a bus 220 joined to an electrode 228 oriented so that the bus 220 faces the viewer. The component labeled 228 in FIG. 20 also corresponds to an electrode joined to a bus and is oriented so that the electrode 228 faces the viewer. In some embodiments, the electrode 228 may be arranged in a shape that mirrors or follows the shape of gasket 170. Processing assembly 200 may include two electrodes 228 that comprise gold that is vacuum deposited onto a thin plastic film. The electrodes 228 may be evenly spaced apart across the chamber 208 and arranged parallel to the opposing electrode.

Processing assembly 200 may include a gasket 170 and spacer that may be received in chamber 208. The gasket 170 forms the chamber 208 shape and determines the volume of the well(s). The gasket 170 forms the liquid-tight seals of the well, and the gasket 170 may form multiple wells. The spacer may be a non-electrically conductive element that supports the shape of the gasket, maintains the distance between the electrodes 228, and maintains the parallelism of the electrodes 228. The gasket 170 may take at least one of several shapes. For example, gasket 170 may have eight wells 172 which may be sized for samples of 50 μL in each well 172. In some embodiments, gasket 170 may be made of silicone rubber or other non-cytotoxic materials. Processing assembly 200 may be configured for use with any gasket size and arrangements described herein such that the processing assembly 200 may be used for any number of sized gaskets 170.

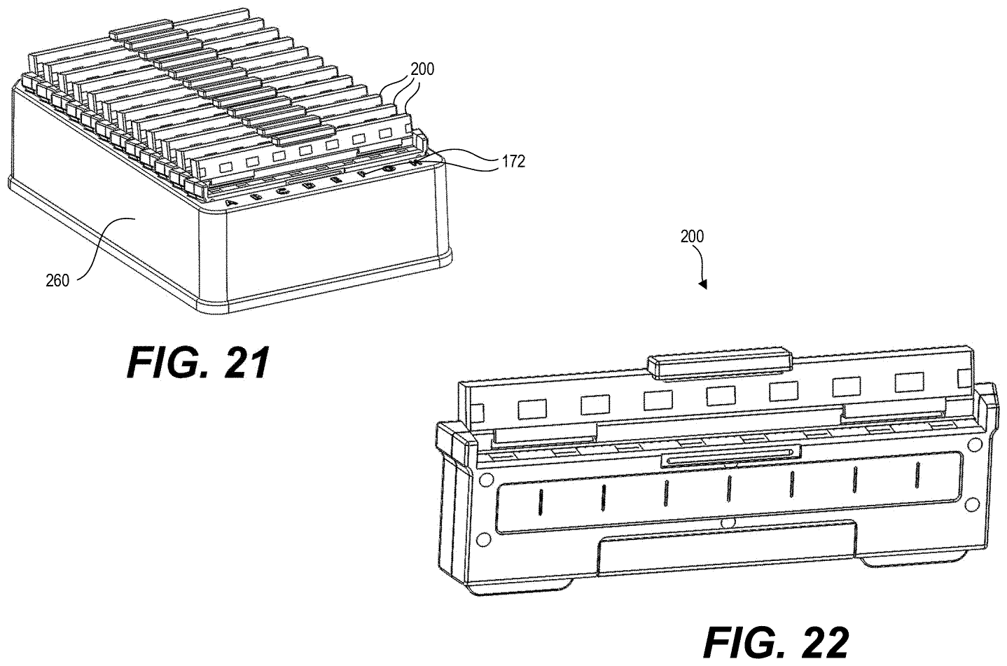

FIG. 21 illustrates a tray 260 configured to receive a plurality of multi-well processing assemblies 260. As illustrated in FIGS. 21 and 22, multi-well processing assemblies may be loaded into tray 260 without lids. Tray 206 may receive twelve processing assemblies 200, and each processing assembly may include eight wells (e.g., wells 172). Accordingly, each tray 206 may include ninety-six wells.

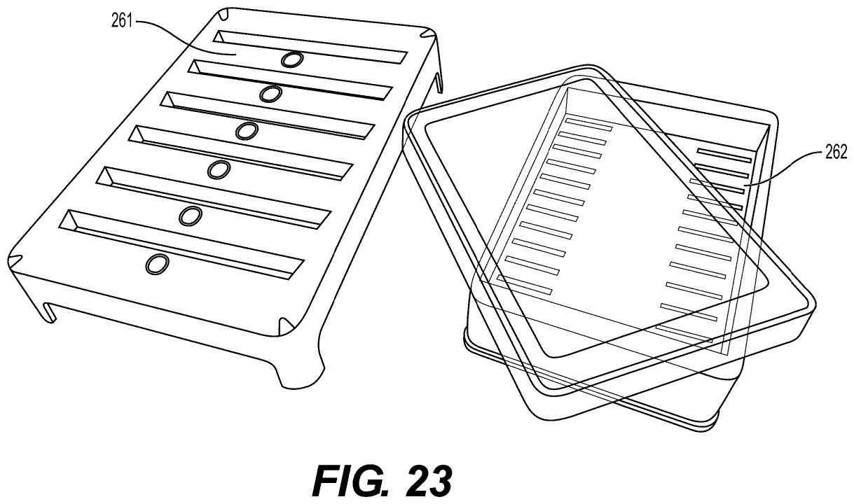

FIG. 23 illustrates a tray 261 configured to receive six processing assemblies 200, which may be used in a manual workflow, and a tray 262 configured to receive twelve processing assemblies, which may include a cover.

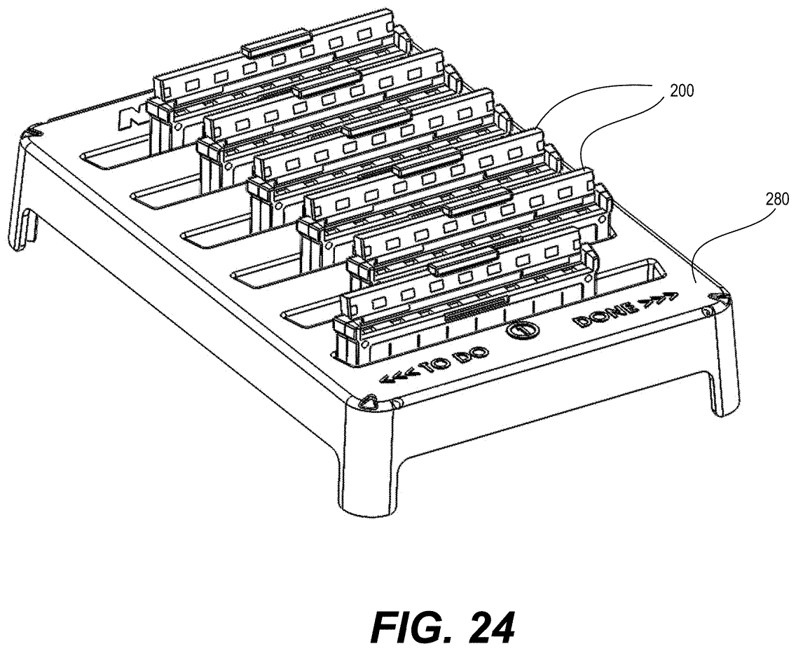

FIG. 24 illustrates a multi-well rack 280 that can receive a plurality of processing assemblies 200 and may provide for loading, unloading, and organization of processing assemblies 200.

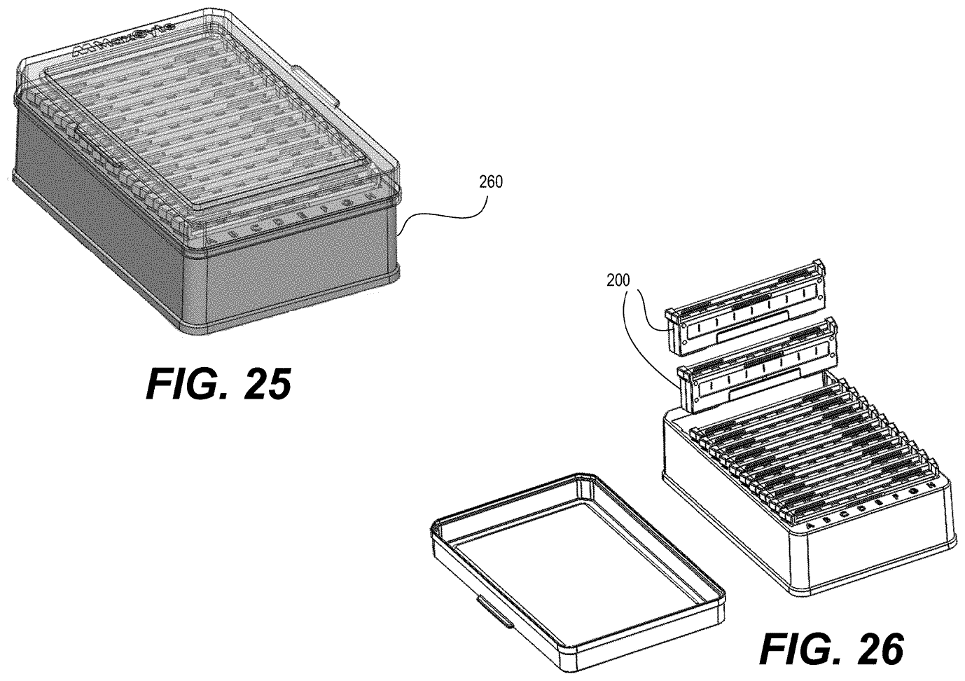

FIGS. 25 and 26 illustrate tray 260 with a lid closure and the loading and unloading of processing assemblies 200 into tray 260.

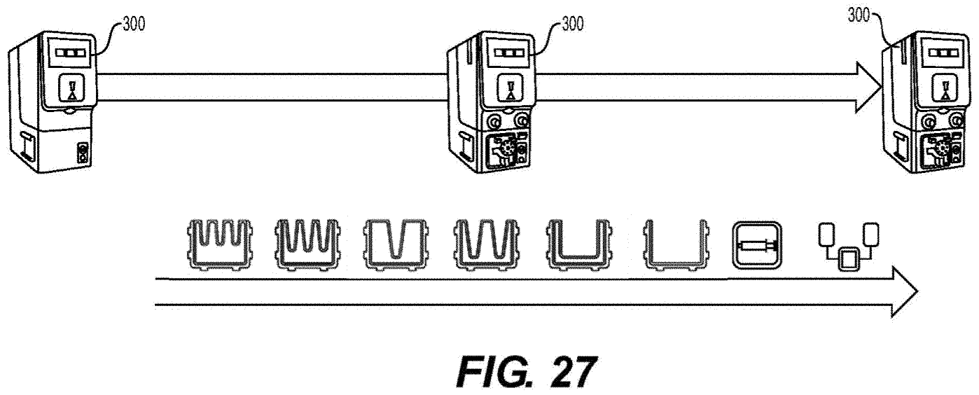

FIG. 27 illustrates exemplary electroporation systems 300 that the disclosed embodiments may be compatible with.

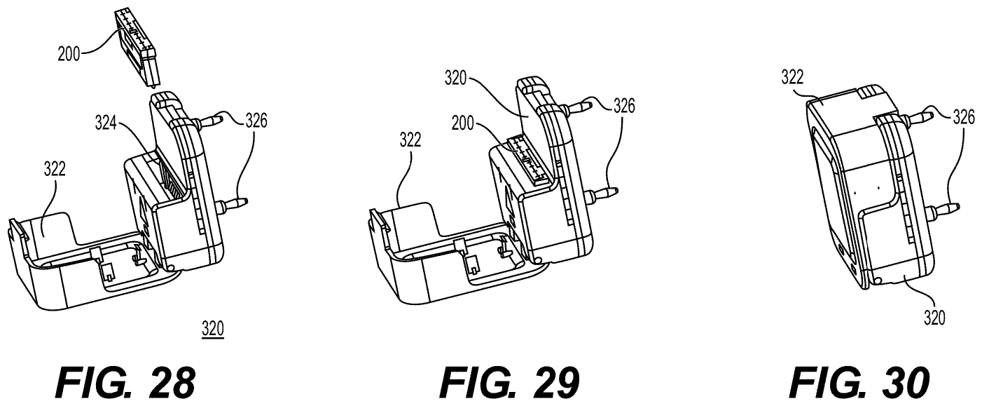

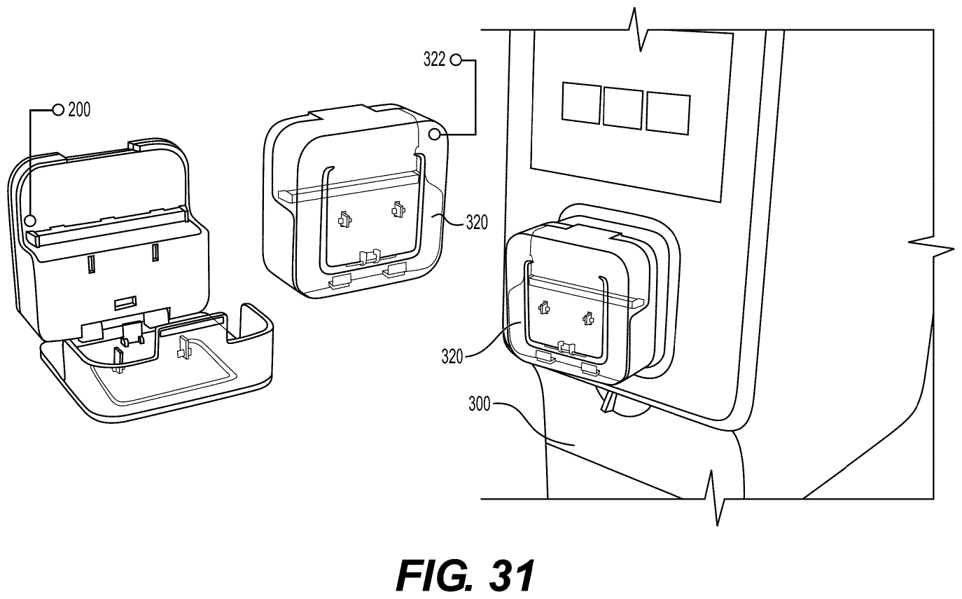

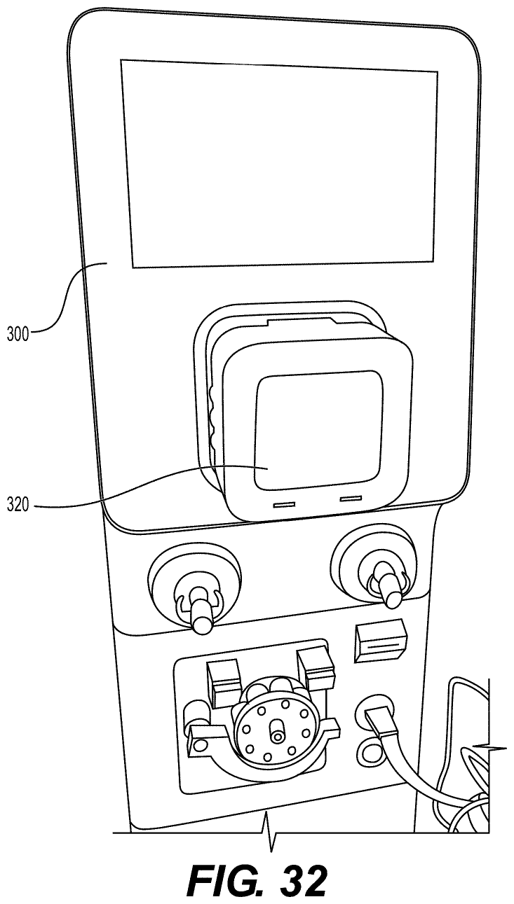

FIGS. 28-32 illustrate a docking station 320 that may connect processing assemblies (e.g., processing assembly 200) to an electroporation system (e.g., electroporation system 300). Docking station 320 may include a lid 322 that may be connected via a hinge connection to docking station 320. Lid 322 may be configured to move between an open position (FIGS. 28 and 29) and a closed position (FIG. 30). Docking station 320 may have a port 324 configured to receive one or more processing assemblies 200. Docking station 320 may also have electrical contacts 326 that may connect to receptacles on an electroporation system (e.g., electroporation system 300).

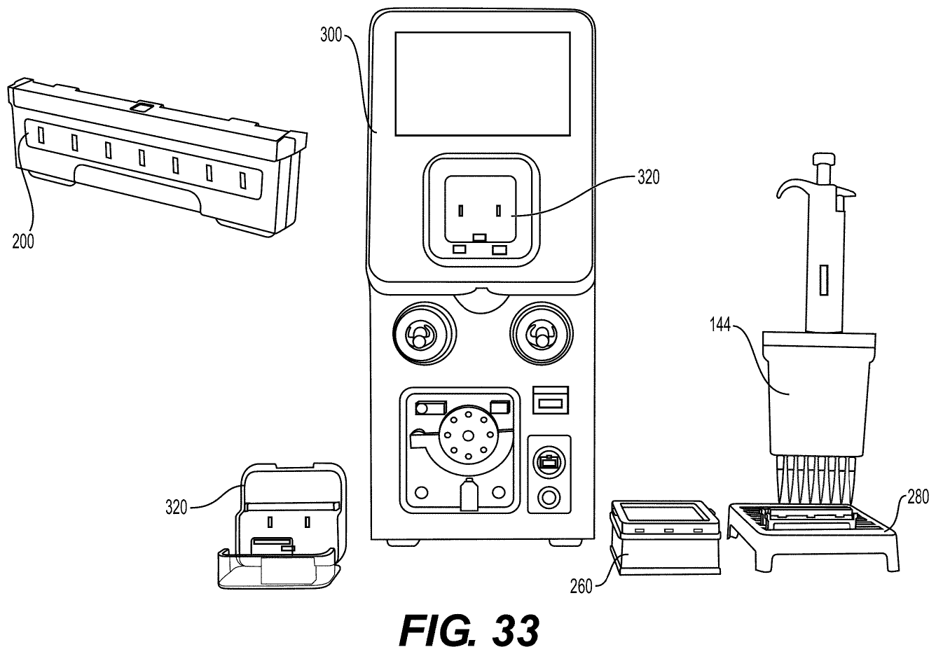

FIG. 33 shows the multi-well processing assembly 200, electroporation system 300, docking station 320, tray 260, loading device 144, and rack 280.

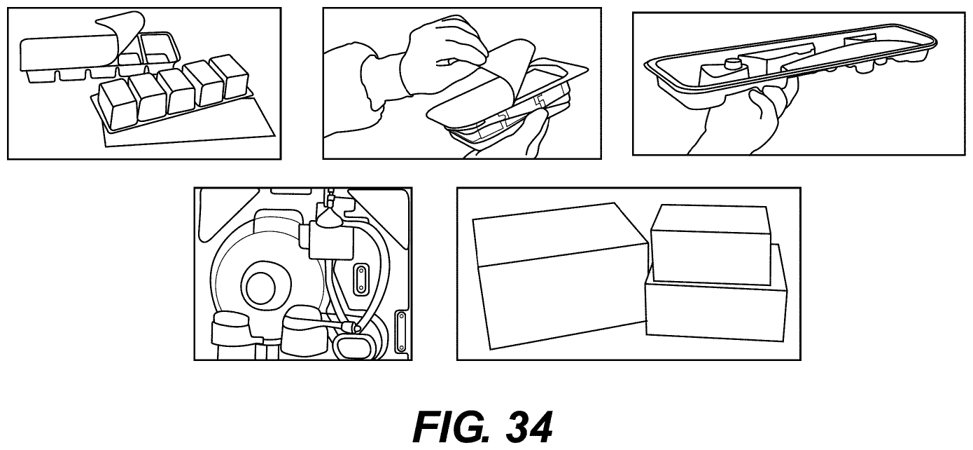

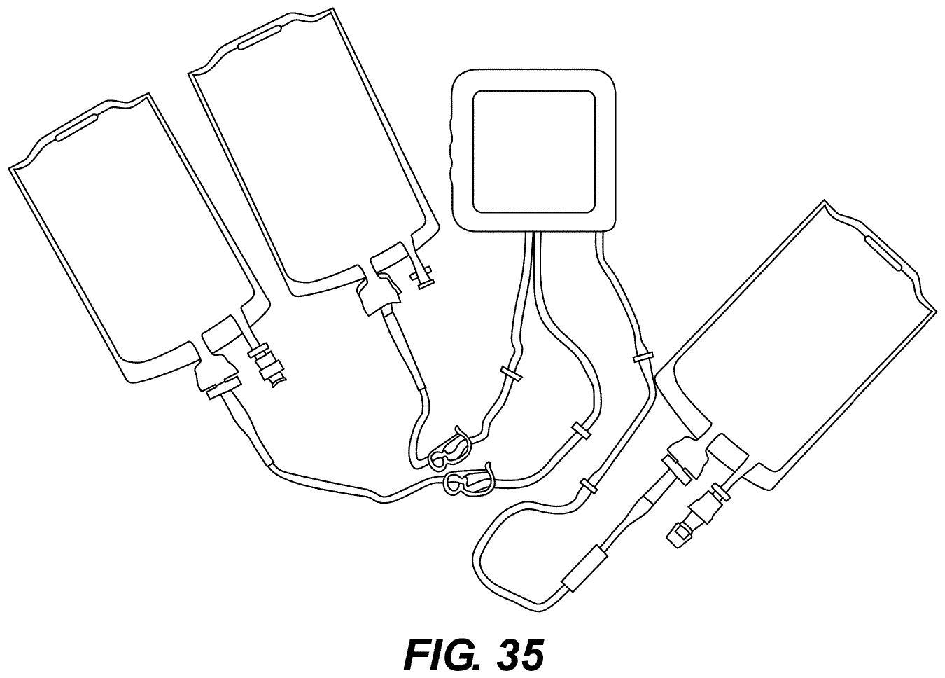

FIG. 34 illustrates a plurality of packaging examples that improve handling of processing assemblies and materials described above, and may allow users to more easily distinguish between Good Manufacturing Processed (GMP) products, and research products.

FIG. 35 illustrates packaging examples including bags having sizes 5-15 mL.

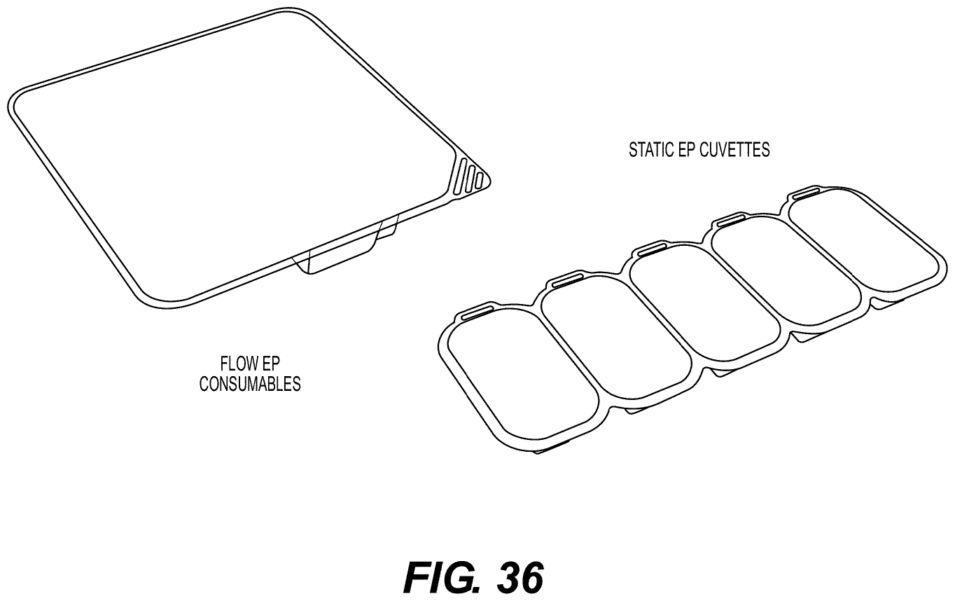

FIG. 36 illustrates packaging examples for flow electroporation consumables and static electroporation cuvettes.

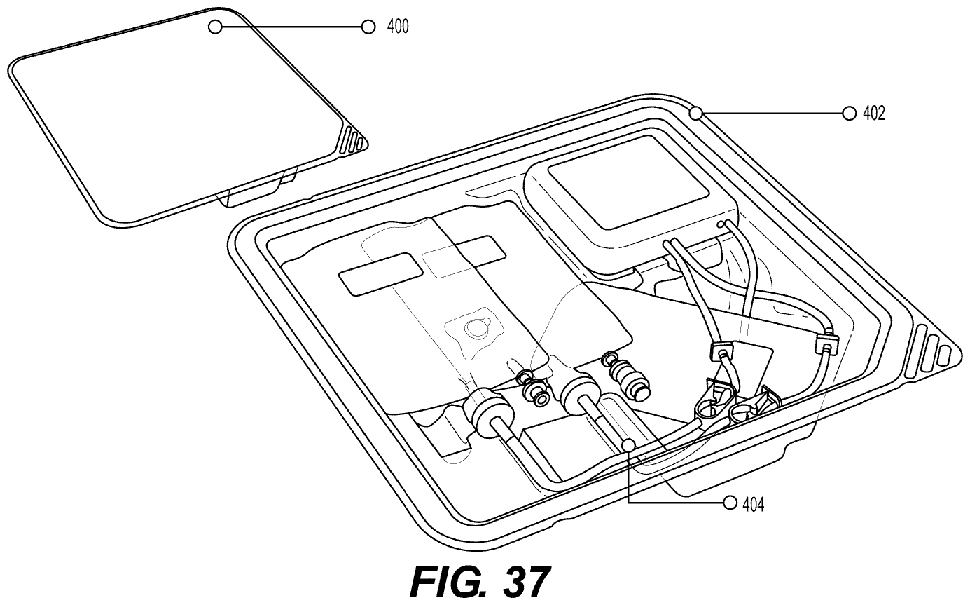

FIG. 37 illustrates packaging examples for flow electroporation consumables. The packaging examples may include a sealed Tyvek cover 400 that may ensure sterility of the package. The packaging examples may also provide a clear thermoformed tray 402 that may protect the contents of the package, provide organization to the contents of the package, and allow for improved transportability. The tray 402 may include guide members 404 that may organize tubes to prevent kinking.

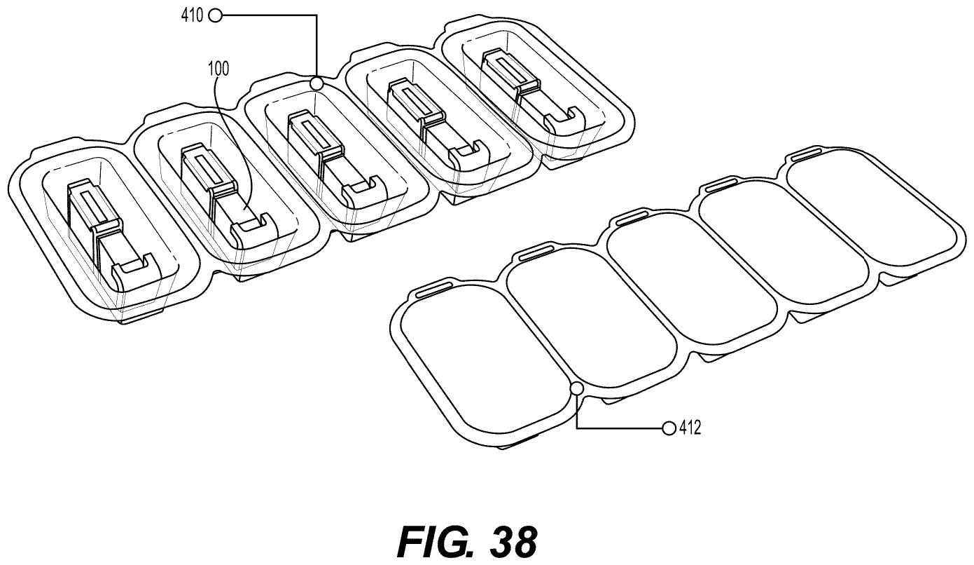

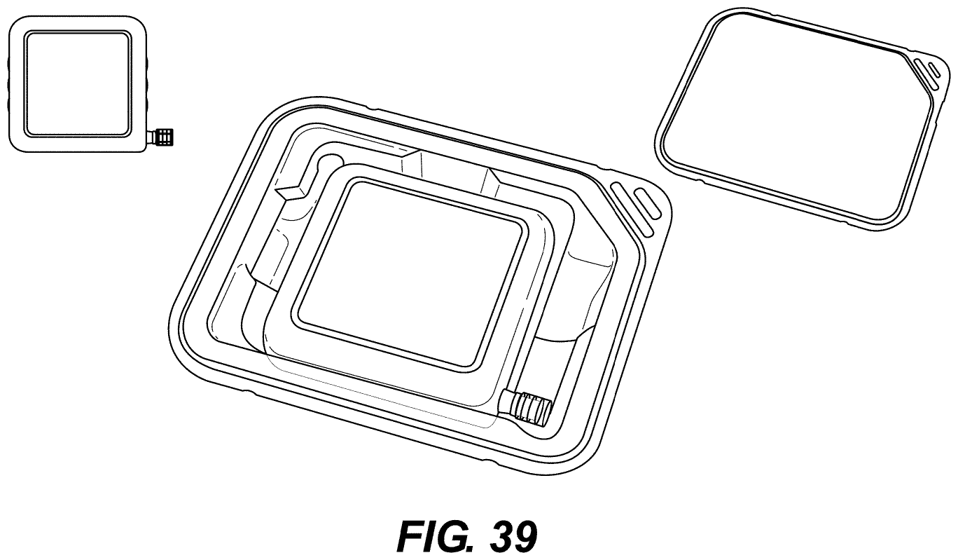

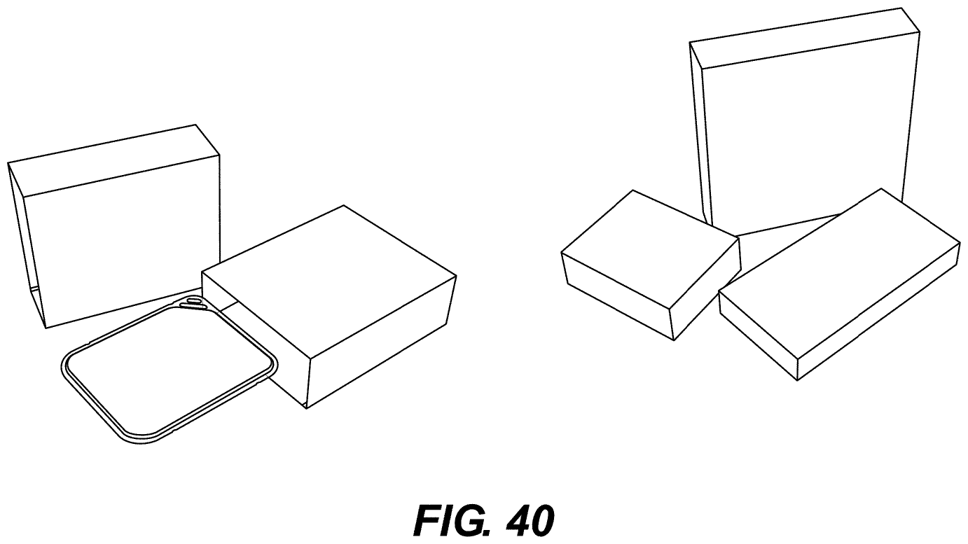

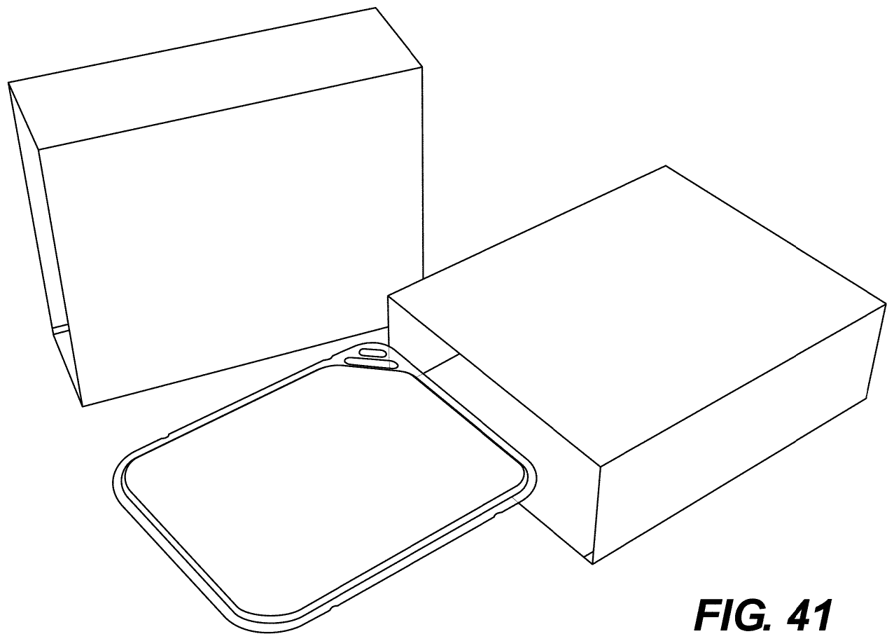

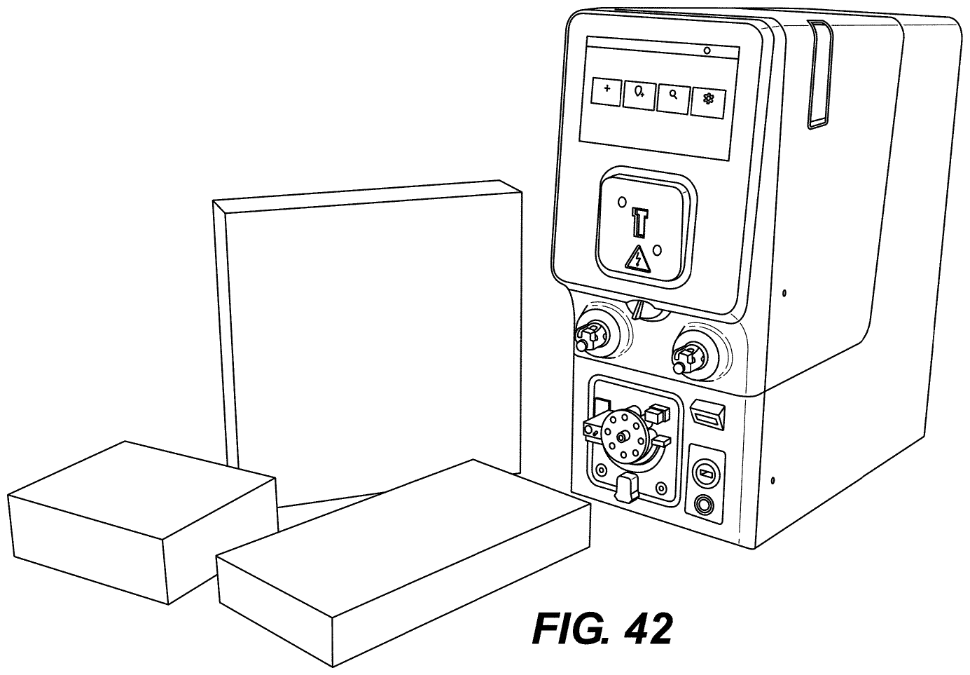

FIG. 38 illustrates packaging examples that may be used for processing assemblies 100. The packaging examples may include a five-position processing assembly tray 410 that may receive processing assemblies 100. The tray 410 may secure and protect each individual processing assembly, allow for stacking and organization of the trays 410, and may provide tear away perforations 412 for individual use.

FIG. 39 illustrates packaging examples for static electroporation processing assemblies.

FIGS and 40-42 illustrate outer packaging for research (RUO) and for GMP products.

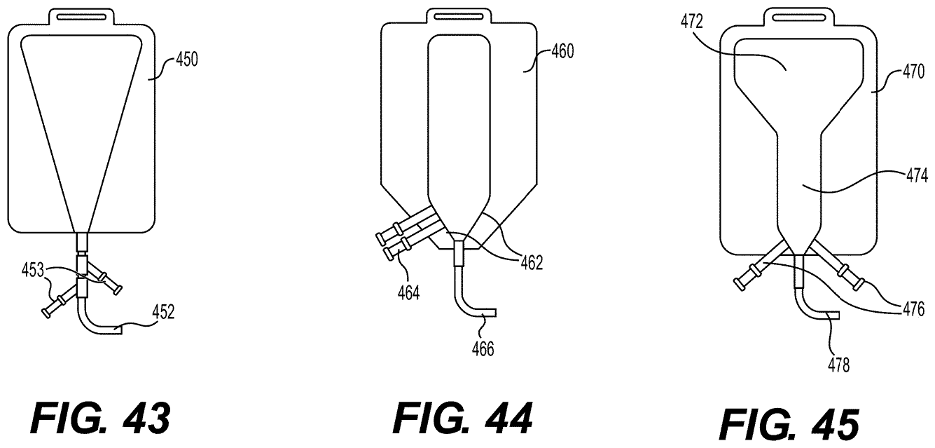

FIGS. 43-45 illustrate exemplary embodiments of bags for use in flow electroporation assemblies. Bag 450 may include a V-shape interior that drains into outlet 452 that may have a plurality of connectors 453.

Bag 460 may include a narrower inner chamber having angled lower surfaces 462, one of the lower surfaces 462 may include one or more connectors 464 and the bag 460 may also include a centrally positioned outlet 466.

Bag 470 may include a wide upper chamber 472 and a narrow lower chamber 474, the lower chamber 474 may include connectors 476 at each angled bottom surface and a centrally positioned outlet 478.

Bags 450, 460, 470 may include Luer fittings, Luer-activated ports, tubing, tube clamps and labels (see diagram in FIGS. 43-45). Bags may be used as a sample bag, a collection bag, and an air bag.

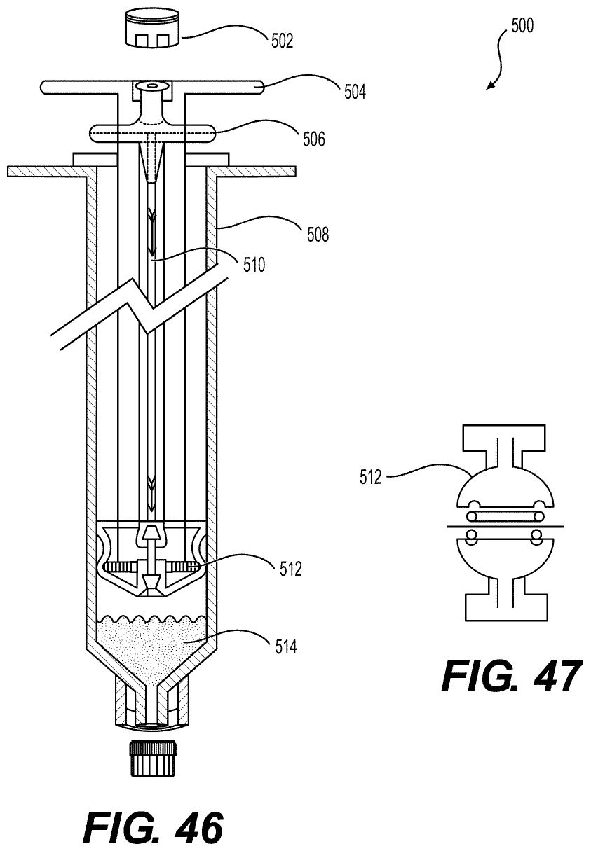

FIGS. 46-49 show a syringe assembly 500 that may be used to load samples into processing assemblies (e.g., processing assembly 100, 200). The syringe assembly 500 may include a Luer cap 502, a plunger 504, a filter stop 506, a syringe barrel 508, an air pathway 510, a plunger seal 512, and may include a cell culture 514. The syringe assembly 500 may reduce a cell loss that may occur in common syringe assemblies.

FIG. 47 shows a detailed view of the plunger seal 512.

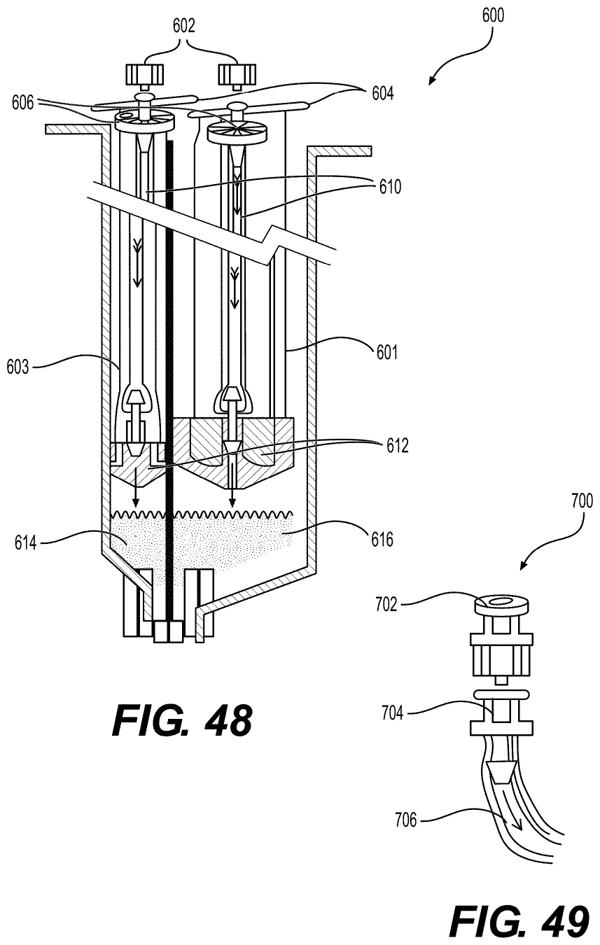

FIG. 48 illustrates a syringe assembly 600 that includes a two-barrel design. The two-barrel design may include a first chamber 601 and a second chamber 603. Each chamber 601, 603 may include a Luer cap 602, a plunger 604, a filter stop 606, an air pathway 610, and a plunger seal 612. The barrels 601, 603 may be different sizes such that one barrel is twice the size of the other barrel. In some embodiments, one barrel 601, 603 may contain a loading agent 614 and the other barrel 601, 603 may include a cell culture 616. The syringe assembly 600 may reduce a cell loss that may occur in common syringe assemblies.

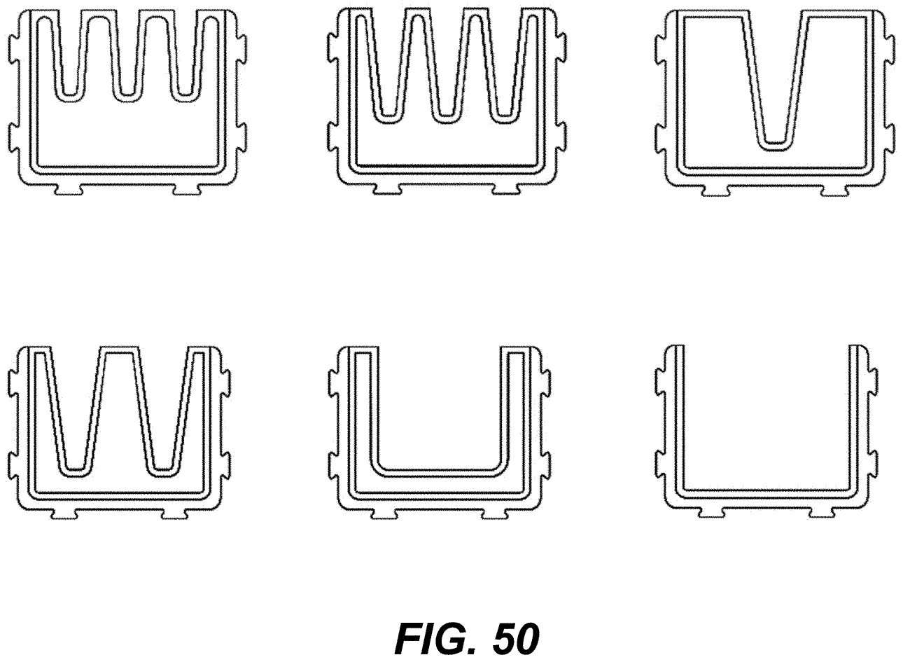

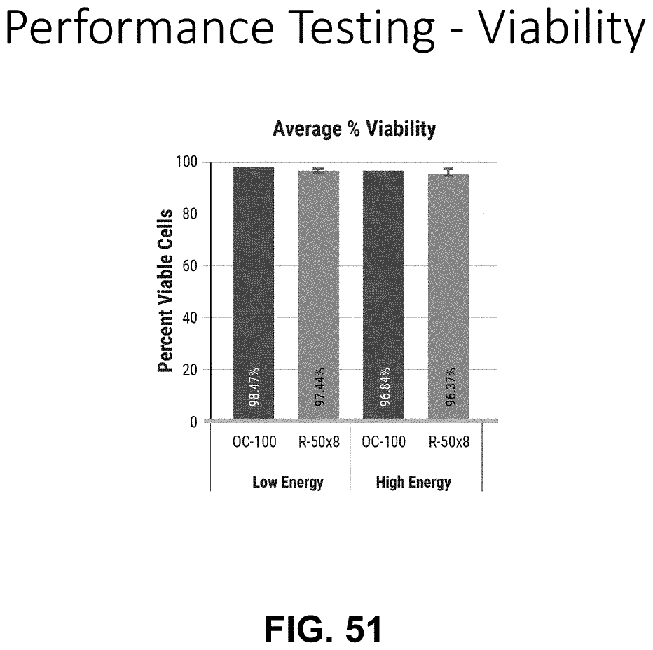

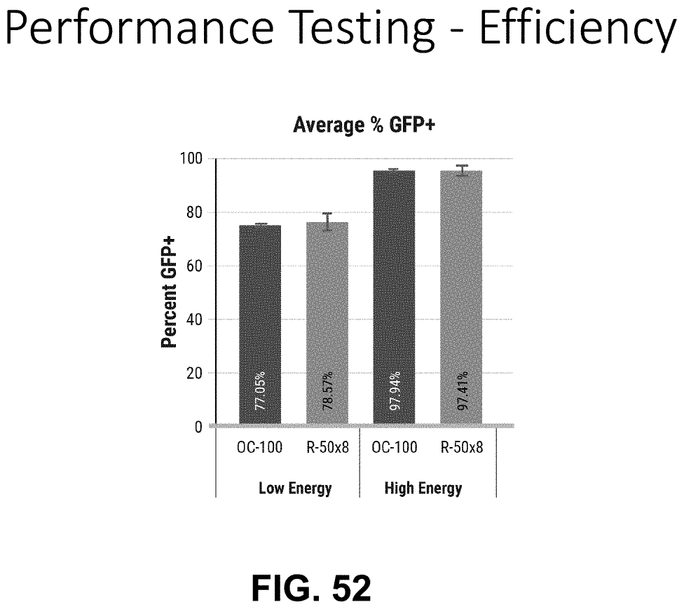

FIG. 49 illustrates a connection assembly 700 that may connect a syringe assembly (e.g., syringe assembly 500, 600) to a chamber (e.g., chamber 108). Connection assembly 700 may include a Luer activated port 702, a Luer barb fitting 704, and tubing to chamber (e.g., chamber 108).

It should be noted that the products and/or processes disclosed may be used in combination or separately. Additionally, exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the prior detailed description be considered as exemplary only, with the true scope and spirit being indicated by the following claims.

The products and/or processes disclosed herein may be used in any application in which electroporation may be useful. Exemplary applications include assay development (such as, e.g., by co-expressing reporter and target proteins in varying ratios, and/or varying subunit ratios), developing animal models of disease, identifying and characterizing potential biomarkers, developing cell-based disease models, assessing the efficacy of pharmacological tool compounds, functional analysis of proteins of interest, in vitro and in vivo genetic manipulation, characterizing disease associated genetics, antibody discovery (such as, e.g., varying heavy/light chain ratios, and/or testing sequence variants), protein antigen and derivative expression (such as, e.g., testing sequence variants, and/or optimizing expression plasmids), gene knockdown (such as, e.g., testing various siRNA sequences and/or concentrations), and developing cell-based assays (such as, e.g., varying report/target ratios and/or relative subunit ratios), and developing therapeutics (such as, e.g., by testing sequence variants of secreted proteins, receptors and other biologics, and optimizing transposon: transposase ratios for non-vial integration of transgenes).

In some embodiments, the geometry of an electroporation chamber may be adjusted to adjust electric field strength. Field strength is calculated using voltage divided by gap size. The geometry of an electroporation chamber can be a function of the distance between electrodes, or “gap size.” Thus, in some embodiments, gap size of electrodes within an electroporation chamber may be controlled to adjust the electric field strength. By increasing the gap size, field strength can be increased without changing voltage. To derive the voltage needed to accomplish electroporation if the desired field strength and gap size are known, field strength (kV) is multiplied by gap size (cm). Electrodes of electroporation chambers can comprise two or more “plate” electrodes. The electrode plate can be addressable with an electric pulse as determined by the present disclosure. The electrodes can comprise an array of between 1 and 100 cathodes and 1 and 100 anodes, there being an even number of cathodes and anodes so as to form pairs of positive and negative electrodes. The plates can comprise a width dimension that is generally greater than the distance, or gap, between opposing electrodes, or greater than twice the gap distance.

The cathode and anode electrodes can be spaced on opposing interior sides of an electroporation chamber such that the electroporation chamber comprises an electrode gap size of at most or at least about 0.001 cm to 10 cm, 0.001 cm to 1 cm, 0.01 cm to 10 cm, 0.01 cm to 1 cm, 0.1 cm to 10 cm, 0.1 cm to 1 cm, 1 cm to 10 cm, or any value from 0.001 cm to 10 cm or range derivable therein. In some embodiments, the electroporation chamber comprises an electrode gap between 0.001 cm and 10 cm, 0.001 cm and 1 cm, 0.01 cm and 10 cm, 0.01 cm and 1 cm, 0.1 cm and 10 cm, 0.1 cm and 1 cm, 1 cm and 10 cm, or any value from 0.001 cm to 10 cm or range derivable therein. In some embodiments, the electroporation chamber comprises an electrode gap between 0.01 cm and 1 cm, any value from 0.01 cm to 1 cm, or any range derivable therein. In some embodiments, the electroporation chamber comprises an electrode gap between 0.4 cm and 1 cm, any value from 0.4 cm to 1 cm, or any range derivable therein. Each pair of said anodes and cathodes can be energized at a load resistance (in Ohms) depending upon the chamber size.

The examples presented herein are for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

What is claimed is:

1. A processing assembly configured for use in an electroporation system, the processing assembly comprising:
a housing;
a lid connected to the housing;
an opening in a top surface of the housing;
an electroporation chamber below the opening in the housing, the electroporation chamber comprising:
(i) a gasket forming the shape of the electroporation chamber and defining the volume of one or more wells within the electroporation chamber; and
(ii) two or more electrodes comprising an electrically conductive, non-cytotoxic metal, wherein the two or more electrodes are positioned on opposing sides of the electroporation chamber; and
wherein the processing assembly comprises two or more electrode buses, each connected to a single electrode to form an electrode-bus subassembly.

2. The processing assembly of claim 1, wherein each bus is configured to form an electrical connection between the electroporation chamber and an electroporation system.

3. The processing assembly of claim 1, wherein the electroporation chamber further comprises a spacer that maintains a distance between the two or more electrodes and arranges the two or more electrodes parallel to each other.

4. The processing assembly of claim 1, comprising two electrodes.

5. The processing assembly of claim 1, wherein the electrically conductive, non-cytotoxic metal is gold.

6. The processing assembly of claim 1, wherein the each of the two or more electrodes comprises gold that is vacuum deposited onto a plastic film.

7. The processing assembly of claim 1, wherein the gasket comprises a non-cytotoxic material.

8. A multi-well processing assembly configured for use in an electroporation system, the multi-well processing assembly comprising:
a housing;
a lid rotationally connectable to the housing;
an opening in a top surface of the housing;
an internal chamber below the opening the housing;
an electroporation chamber below the opening in the housing, the electroporation chamber comprising:
a gasket forming the shape of the electroporation chamber and defining the volume of one or more wells within the electroporation chamber; and
two or more electrodes comprising an electrically conductive, non-cytotoxic metal, wherein the two or more electrodes are positioned on opposing sides of the electroporation chamber; and
two or more electrode buses, each connected to a single electrode to form an electrode-bus subassembly.

9. The processing assembly of claim 8, wherein the bus is configured to form an electrical connection between the processing assembly and an electroporation system.

10. The processing assembly of claim 8, wherein the electroporation chamber further comprises a spacer that maintains a distance between the two or more electrodes and arranges the two or more electrodes parallel to each other.

11. The processing assembly of claim 8, comprising two electrodes.

12. The processing assembly of claim 8, wherein the electrically conductive, non-cytotoxic metal is gold.

13. The processing assembly of claim 8, wherein the each of the two or more electrodes comprises gold that is vacuum deposited onto a plastic film.

14. The processing assembly of claim 8, wherein the gasket comprises a non-cytotoxic material.

15. A docking station configured for use in an electroporation system, the docking station comprising:
a housing;
a port in the housing configured to receive one or more processing assemblies;
a lid connected to the housing;
one or more contacts configured to connect the docking station to an electroporation system.

16. An electroporation system comprising:
a processing assembly configured for use in an electroporation system, the processing assembly comprising:
a housing;
a lid rotationally connectable to the housing;
an opening in a top surface of the housing;
an electroporation chamber below the opening in the housing, wherein the electroporation chamber comprises;
(i) two or more electrodes comprising an electrically conductive, non-cytotoxic metal, wherein the two or more electrodes are positioned on opposing sides of the electroporation chamber; and
(ii) a gasket forming the shape of the electroporation chamber and defining the volume of one or more wells within the electroporation chamber;
a docking station, the docking station comprising:
a housing;
a port in the housing configured to receive the processing assembly;
a lid connected to the housing;
one or more contacts configured to connect the docking station to an electroporation system housing.
